(19) United States
(12) Patent Application Publication
(10) Pub. No.: US 2020/0197008 A1
(43) Pub. Date: Jun. 25, 2020

(54) PNEUMATIC DEVICE FOR OPENING AND CLOSING A WOUND

(71) Applicant: KCI Licensing, Inc., San Antonio, TX (US)

(72) Inventors: Benjamin A. Pratt, Poole (GB); Christopher B. Locke, Bournemouth (GB)

(73) Assignee: KCI Licensing, Inc., San Antonio, TX (US)

(21) Appl. No.: 16/639,994

(22) PCT Filed: Aug. 9, 2018
(86) PCT No.: PCT/US2018/045967

§ 371 Date: Feb. 18, 2020

Related U.S. Application Data
(60) Provisional application No. 62/548,716, filed on Aug. 22, 2017.

(51) Int. Cl. A61B 17/08

(52) U.S. Cl. CPC A61B 17/085; A61B 2017/00544

(57) ABSTRACT

A device includes a plurality of pneumatic modules and a pressure control device. The pneumatic modules are configured to attach to a patient's skin along edges of a wound and to translate a pneumatic pressure into a linear force. Each pneumatic module is configured to move between an extended position and a contracted position based on the pneumatic pressure within the pneumatic module. The pressure control device is configured to be pneumatically coupled to each of the pneumatic modules and configured to alternate the pneumatic modules between a first state and a second state. In the first state, the pneumatic pressure within a first set of the pneumatic modules is less than the pneumatic pressure within a second set of the pneumatic modules. In the second state, the pneumatic pressure within the first set of the pneumatic modules is greater than the pneumatic pressure within the second set of the pneumatic modules.

### CROSS-REFERENCE TO RELATED APPLICATIONS

This application claims the benefit of priority to U.S. Provisional Application No. 62/548,716, filed on Aug. 22, 2017, which is incorporated herein by reference in its entirety.

### BACKGROUND

The present disclosure relates generally to a pneumatic device for opening and closing a wound. The present disclosure relates more particularly to a pneumatic device configured to attach to edges of a wound and pull the edges of the wound together or push the edges of the wound apart upon application of pneumatic pressure.

Negative pressure wound therapy (NPWT) has been proven to be very effective on a wide range of wound types by promoting granulation. However, in some instances, it is first necessary to reduce the size of the wound by attempting to get the edges as close as possible. Wound therapy may be most effective when wound size is reduced as much as possible using existing, healthy tissue. Wound closure is traditionally achieved using sutures and staples, among other methods. However, the wound edges can sometimes be too far apart for these techniques to be successful. Additionally, sutures and staples are fixed and do not allow the edges of the wound to be gradually moved without repeated manual intervention by skilled caregivers.

In some instances, it may be necessary to open up a wound to prevent further trauma to the patient. For example, abdominal compartment syndrome typically requires an abdominal wound to be created to relieve pressure within the abdomen. Once pressure is relieved in these cases, the next step is closure of the clinician-made wound. To promote effective healing, the edges of the wound are closed as accurately as possible and as much of the wound closed with existing healthy tissue. For the best aesthetic outcome, the edges should be restored to their original positions.

In cases where mechanical devices, staples, or sutures are used to hold the edges of a wound together during healing, breakdown of healthy tissue can occur at the point of fixture. This is as a result of pressure and stress within the tissue around the fixtures that is not relieved and is therefore constant for a prolonged period of time. It would be desirable to provide a controlled method of opening and closing wounds to ensure the best possible outcome by utilizing healthy tissue for closure, without causing the further breakdown of surrounding tissue.

### SUMMARY

One implementation of the present disclosure is a device including a plurality of pneumatic modules and a pressure control device. The plurality of pneumatic modules are configured to attach to a patient's skin along edges of a wound and to translate a pneumatic pressure into a linear force. Each pneumatic module is configured to move between an extended position and a contracted position based on the pneumatic pressure within the pneumatic module. The pressure control device is configured to be pneumatically coupled to each of the pneumatic modules and configured to alternate the plurality of pneumatic modules between a first state and a second state. In the first state, the pneumatic pressure within a first set of the pneumatic modules is less than the pneumatic pressure within a second set of the pneumatic modules. In the second state, the pneumatic pressure within the first set of the pneumatic modules is greater than the pneumatic pressure within the second set of the pneumatic modules.

In some embodiments, the pressure control device is configured to apply a different pneumatic pressure to each of the plurality of pneumatic modules. In some embodiments, the pressure control device is configured to be pneumatically coupled to a space between the wound and a wound dressing and configured to apply a reduced pressure within the space between the wound and the wound dressing.

In some embodiments, the first set of pneumatic modules and the second set of pneumatic modules are arranged in an alternating sequence along the edges of the wound. In some embodiments, the first set of pneumatic modules and the second set of pneumatic modules are arranged along the edges of the wound such that each pneumatic module in the first set is separated from each other pneumatic module in the first set by at least one pneumatic module in the second set.

In some embodiments, reducing the pneumatic pressure within a pneumatic module causes the pneumatic module to move toward the contracted position and increasing the pneumatic pressure within a pneumatic module causes the pneumatic module to move toward the extended position.

In some embodiments, reducing the pneumatic pressure within a pneumatic module increases the linear force exerted by the pneumatic module and is configured to increase a stress applied to the patient's skin by the pneumatic module. In some embodiments, increasing the pneumatic pressure within a pneumatic module relaxes the linear force exerted by the pneumatic module and is configured to reduce a stress applied to the patient's skin by the pneumatic module.

In some embodiments, one or more of the pneumatic modules includes a relief valve configured to vent the pneumatic module when a difference between ambient pressure and the pneumatic pressure within the pneumatic module exceeds a relief pressure threshold. In some embodiments, the device includes a plurality of pneumatic tubes connecting the pressure control device to the plurality of pneumatic modules.

In some embodiments, each pneumatic module includes a first node configured to attach to the patient's skin along a first edge of the wound, a second node configured to attach to the patient's skin along a second edge of the wound, and a pneumatic connector coupled to the first node and the second node and forming a pneumatic connection between the first node and the second node. The pneumatic connector may be configured to extend and contract based on the pneumatic pressure within the pneumatic module.

In some embodiments, each node includes an adhesive pad configured to adhere the node to the patient's skin. In some embodiments, each node comprises a deep tissue hook configured to attach the node to the patient's skin.

In some embodiments, each node includes an internal volume pneumatically connected to the patient's skin and maintained at an internal pressure less than an external pressure outside the node. In some embodiments, each node is held to the patient's skin by a pressure differential between the internal pressure and the external pressure.

In some embodiments, one or more of the pneumatic modules includes an interlocking feature configured to engage when a separation distance between the first node and the second node reaches a minimum distance threshold and to prevent the separation distance from decreasing below the minimum distance threshold. In some embodiments, the interlocking feature is adjustable to allow the minimum distance threshold to be increased or decreased.

Another implementation of the present disclosure is a device including a plurality of pneumatic modules and a pressure control device. Each pneumatic module may include a first node configured to attach to a patient's skin along a first edge of the wound, a second node configured to attach to the patient's skin along a second edge of the wound, and a pneumatic connector coupled to the first node and the second node and forming a pneumatic connection between the first node and the second node. The pneumatic connector may be configured to extend and contract based on a pneumatic pressure within the pneumatic module. The pressure control device may be configured to be pneumatically coupled to each of the pneumatic modules and configured to alternate the plurality of pneumatic modules between a first state and a second state. In the first state, the pneumatic pressure within a first set of the pneumatic modules is less than the pneumatic pressure within a second set of the pneumatic modules. In the second state, the pneumatic pressure within the first set of the pneumatic modules is greater than the pneumatic pressure within the second set of the pneumatic modules.

In some embodiments, each node includes an adhesive pad configured to adhere the node to the patient's skin. In some embodiments, each node comprises a deep tissue hook configured to attach the node to the patient's skin.

In some embodiments, each node includes an internal volume pneumatically connected to the patient's skin and maintained at an internal pressure less than an external pressure outside the node. In some embodiments, each node is held to the patient's skin by a pressure differential between the internal pressure and the external pressure.

In some embodiments, one or more of the pneumatic modules includes an interlocking feature configured to engage when a separation distance between the first node and the second node reaches a minimum distance threshold and to prevent the separation distance from decreasing below the minimum distance threshold. In some embodiments, the interlocking feature is adjustable to allow the minimum distance threshold to be increased or decreased.

In some embodiments, the pressure control device is configured to apply a different pneumatic pressure to each of the plurality of pneumatic modules. In some embodiments, the pressure control device is pneumatically coupled to a space between the wound and a wound dressing and configured to apply a reduced pressure within the space between the wound and the wound dressing.

In some embodiments, the first set of pneumatic modules and the second set of pneumatic modules are arranged in an alternating sequence along the edges of the wound. In some embodiments, the first set of pneumatic modules and the second set of pneumatic modules are arranged along the edges of the wound such that each pneumatic module in the first set is separated from each other pneumatic module in the first set by at least one pneumatic module in the second set.

In some embodiments, reducing the pneumatic pressure within a pneumatic module causes the pneumatic connector to contract. In some embodiments, increasing the pneumatic pressure within a pneumatic module causes the pneumatic connector to extend.

In some embodiments, reducing the pneumatic pressure within a pneumatic module increases a linear force exerted by the pneumatic module and is configured to increase a stress applied to the patient's skin by the pneumatic module. In some embodiments, increasing the pneumatic pressure within a pneumatic module relaxes a linear force exerted by the pneumatic module and is configured to reduce a stress applied to the patient's skin by the pneumatic module.

In some embodiments, one or more of the pneumatic modules includes a relief valve configured to vent the pneumatic module when a difference between ambient pressure and the pneumatic pressure within the pneumatic module exceeds a relief pressure threshold. In some embodiments, the device includes a plurality of pneumatic tubes connecting the pressure control device to the plurality of pneumatic modules.

Another implementation of the present disclosure is a device including a first foam clamp configured to attach to a patient's skin along a first edge of the wound, a second foam clamp configured to attach to the patient's skin along a second edge of the wound, a foam material disposed between and coupled to the first foam clamp and the second foam clamp. The foam material may be longitudinally collapsible to draw the first and second foam clamps toward each other upon application of a negative pressure.

In some embodiments, the foam material is longitudinally expandable to push the first and second foam clamps away from each other upon application of a positive pressure. In some embodiments, the foam material is configured to return to a non-collapsed state and push the first and second foam clamps away from each other upon removal of the negative pressure.

In some embodiments, the device includes a pressure control device pneumatically coupled to the foam material and configured to control a pressure within the foam material to cause the foam material to draw the first and second foam clamps toward each other.

In some embodiments, the device includes an impermeable layer along an outer surface of the foam material and configured to prevent airflow between the foam material and an ambient environment. In some embodiments, the impermeable layer is a polyurethane film. In some embodiments, the device includes an impermeable layer along an wound-facing surface of the foam material and configured to prevent airflow between the foam material and the wound.

In some embodiments, the foam material is pneumatically coupled to a space between the wound and a wound dressing such that the pneumatic pressure within the foam material is applied to the space between the wound and the wound dressing.

In some embodiments, the foam clamps comprise conduits pneumatically connected to the patient's skin and maintained at an internal pressure less than an ambient pressure. In some embodiments, the foam clamps are held to the patient's skin by a pressure differential between the internal pressure and the ambient pressure.

In some embodiments, each foam clamp includes an adhesive pad configured to adhere the foam clamp to the patient's skin. In some embodiments, each foam clamp comprises a deep tissue hook configured to attach the foam clamp to the patient's skin.

In some embodiments, one or more of the foam clamps includes an interlocking feature configured to engage when a separation distance between the first foam clamp and the second foam clamp reaches a minimum distance threshold and to prevent the separation distance from decreasing below the minimum distance threshold. In some embodiments, the interlocking feature is adjustable to allow the minimum distance threshold to be increased or decreased.

In some embodiments, reducing the pressure within the foam material causes the foam material to draw the first and second foam clamps toward each other. In some embodiments, increasing the pressure within the foam material causes the foam material to push the first and second foam clamps away from each other.

In some embodiments, reducing the pressure within the foam material increases a force exerted by the foam material on the foam clamps and is configured to increase a stress applied to the patient's skin by the foam clamps. In some embodiments, increasing the pressure within the foam material relaxes a force exerted by the foam material on the foam clamps and is configured to reduce a stress applied to the patient's skin by the foam clamps.

In some embodiments, the device includes a relief valve configured to vent the foam material when a difference between ambient pressure and the pressure within the foam material exceeds a relief pressure threshold.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a drawing of a pneumatic device for opening and closing a wound including a plurality of pneumatic modules and a pressure control device, according to an exemplary embodiment.

FIG. 2 is drawing illustrating one of the pneumatic modules of FIG. 1 in greater detail, according to an exemplary embodiment.

FIG. 3 is a cross-sectional view of the pneumatic module of FIG. 2, according to an exemplary embodiment.

FIG. 4 is a drawing of the pneumatic module of FIG. 2 in a contracted position, according to an exemplary embodiment.

FIG. 5 is another drawing of the pneumatic device of FIG. 1 with several of the pneumatic modules in a partially-contracted position, according to an exemplary embodiment.

FIG. 6 is another drawing of the pneumatic device of FIG. 1 with a first set of the pneumatic modules in a contracted position and a second set of the pneumatic modules in a relaxed position, according to an exemplary embodiment.

FIG. 7 is another drawing of the pneumatic device of FIG. 1 with all of the pneumatic modules in a contracted position, according to an exemplary embodiment.

FIG. 8 is another drawing illustrating one of the pneumatic modules of FIG. 1 in greater detail, according to an exemplary embodiment.

FIG. 9 is a drawing of a manifold which can be used to pneumatically couple the pressure control device to the plurality of pneumatic modules, according to an exemplary embodiment.

FIG. 10 is a drawing of a collapsible foam pneumatic module in a relaxed position, according to an exemplary embodiment.

FIG. 11 is a drawing of the collapsible foam pneumatic module of FIG. 10 in a stretched position, according to an exemplary embodiment.

FIG. 12 is an exploded view drawing of the collapsible foam pneumatic module of FIG. 10, according to an exemplary embodiment.

FIG. 13 is a cross-sectional view of the collapsible foam pneumatic module of FIG. 10, according to an exemplary embodiment.

FIG. 14 is a drawing of a plurality of the collapsible foam pneumatic modules of FIG. 10 applied to a wound, according to an exemplary embodiment.

FIG. 15 is another drawing of a plurality of the collapsible foam pneumatic modules of FIG. 10 applied to a wound with pneumatic tubes attached to the pneumatic modules, according to an exemplary embodiment.

FIG. 16 is another drawing of a plurality of the collapsible foam pneumatic modules of FIG. 10 applied to a wound with a wound dressing over the pneumatic modules, according to an exemplary embodiment.

### DETAILED DESCRIPTION

### Overview

Referring generally to the FIGURES, a pneumatic device for opening and closing a wound and components thereof are shown, according to various exemplary embodiments. The pneumatic device includes a plurality of pneumatic modules and a pressure control device. Each of pneumatic modules may include a first node, a second node, and a pneumatic connector. The first node may attach to a patient's skin along a first edge of a wound, whereas the second node may attach to the patient's skin along a second edge of wound. The pneumatic connector may be coupled both nodes and may form a pneumatic connection between the nodes.

Each of the pneumatic modules can translate a pneumatic pressure into a linear force. The linear forces created by the pneumatic modules may cause the edges of the wound to be pulled toward each other to close the wound or pushed apart from each other to open the wound. For example, each pneumatic connector can be configured to extend and contract based on the pneumatic pressure within the corresponding pneumatic module. When the pressure within a pneumatic module increases, the pneumatic connector may extend, thereby creating a linear force between the nodes which pushes the nodes apart from each other. Conversely, when the pressure within a pneumatic module decreases, the corresponding pneumatic connector may contract, thereby creating a linear force between the nodes which pulls the nodes toward each other.

The pressure control device can be configured to gradually reduce the pressure within each pneumatic module to draw the nodes closer together and facilitate wound closure. If the wound needs to be reopened, the pressure control device can increase the pressure within each pneumatic module to push the nodes apart and reopen the wound. The pneumatic device can be used to both open and close a wound (e.g., an abdominal wound) in a controlled manner during ongoing care without the need for sutures.

The pressure control device can be configured to alternate the pneumatic modules between a first state and a second state. In the first state, the pneumatic pressure within a first set of the pneumatic modules may be less than the pneumatic pressure within a second set of the pneumatic modules. In the second state, the pneumatic pressure within the first set of the pneumatic modules may be greater than the pneumatic pressure within the second set of the pneumatic modules. The different pneumatic pressures within each set of pneumatic modules may cause some pneumatic modules to expand or relax while other pneumatic modules contract. Advantageously, alternating the pneumatic modules between the first state and the second state may prevent skin breakdown by intermittently relaxing the stress applied to the patient's skin at the location of each pneumatic module. These and other features and advantages of the pneumatic device are described in detail below.

### Pneumatic Device

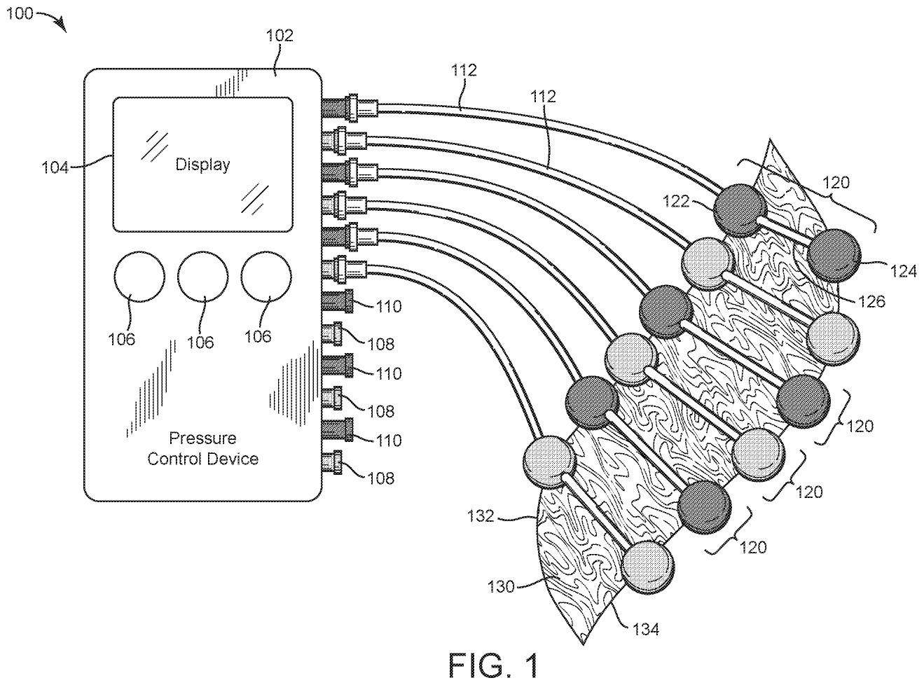

Referring now to FIG. 1, a pneumatic device 100 for opening and closing a wound is shown, according to an exemplary embodiment. Pneumatic device 100 is shown to include a plurality of pneumatic modules 120 and a pressure control device 102. Each of pneumatic modules 120 may include a first node 122, a second node 124, and a pneumatic connector 126. First node 122 can be configured to attach to a patient's skin along a first edge 132 of a wound 130. Similarly, second node 124 can be configured to attach to the patient's skin along a second edge 132 of wound 130. Nodes 122-124 can be attached to the patient's skin using adhesive pads, deep tissue hooks, negative pressure, or other means. Pneumatic connector 126 can be coupled to first node 122 and second node 124 and may form a pneumatic connection between first node 122 and second node 124.

Each of pneumatic modules 120 can be configured to translate a pneumatic pressure into a linear force. For example, each pneumatic connector 126 can be configured to extend and contract based on the pneumatic pressure within the corresponding pneumatic module 120. When the pressure within a pneumatic module 120 increases, the corresponding pneumatic connector 126 may extend, thereby creating a linear force between nodes 122-124 which pushes nodes 122-124 apart from each other. Conversely, when the pressure within a pneumatic module 120 decreases, the corresponding pneumatic connector 126 may contract, thereby creating a linear force between nodes 122-124 which pulls nodes 122-124 toward each other. The linear forces created by pneumatic modules 120 may cause edges 132-134 to be pulled toward each other to close wound 130 or pushed apart from each other to open wound 130.

Pneumatic modules 120 can be pneumatically coupled to pressure control device 102 via pneumatic tubes 112. For example, pressure control device 102 is shown to include a plurality of pneumatic ports 108-110. Each pneumatic tube 112 may releasably attach to one of pneumatic ports 108-110 and to one of pneumatic modules 120. Pneumatic tubes 112 may pneumatically couple ports 108-110 to pneumatic modules 120 such that the pneumatic pressure within each pneumatic module 120 is equal to the pneumatic pressure at the corresponding port 108-110 to which the pneumatic module 120 is coupled.

Pressure control device 102 can be configured to monitor and control the pneumatic pressure at each of pneumatic ports 108-110. For example, pressure control device 102 can include one or more internal air pumps, valves, or other components which can be operated to adjust the pneumatic pressure at each of pneumatic ports 108-110. Pressure control device 108 may include one or more pressure sensors configured to measure the pneumatic pressure at each of pneumatic ports 108-110. In some embodiments, pressure control device 102 includes an electronic display 104 and one or more control buttons 106. A user can view the pneumatic pressure applied to each of pneumatic modules 120 via electronic display 104 and can adjust the pneumatic pressure via control buttons 106.

Pressure control device 102 can be configured to gradually reduce the pressure within each pneumatic module 120 to draw nodes 122-124 closer together and facilitate wound closure. If wound 130 needs to be reopened, pressure control device 102 can increase the pressure within each pneumatic module 120 to push nodes 122-124 apart and reopen wound 130. Pressure control device 102 can be used to both open and close wound 130 (e.g., an abdominal wound) in a controlled manner during ongoing care without the need for sutures.

Pressure control device 102 is shown to include two sets of pneumatic ports 108 and 110. In some embodiments, pressure control device 102 is configured to independently control the pneumatic pressure at each set of ports 108 and 110. For example, pressure control device 102 can simultaneously apply a first pneumatic pressure at ports 108 and a second pneumatic pressure, different from the first pneumatic pressure, at ports 110. In some embodiments, pressure control device 102 is configured to independently control the pneumatic pressure at each of ports 108-110 such that each individual port 108-110 can output a different pneumatic pressure. In other embodiments, pressure control device 102 groups pneumatic ports 108-110 into multiple groups and outputs a different pneumatic pressure to each group of ports 108-110. The operation of pressure control device 102 is described in greater detail below.

### Pneumatic Module

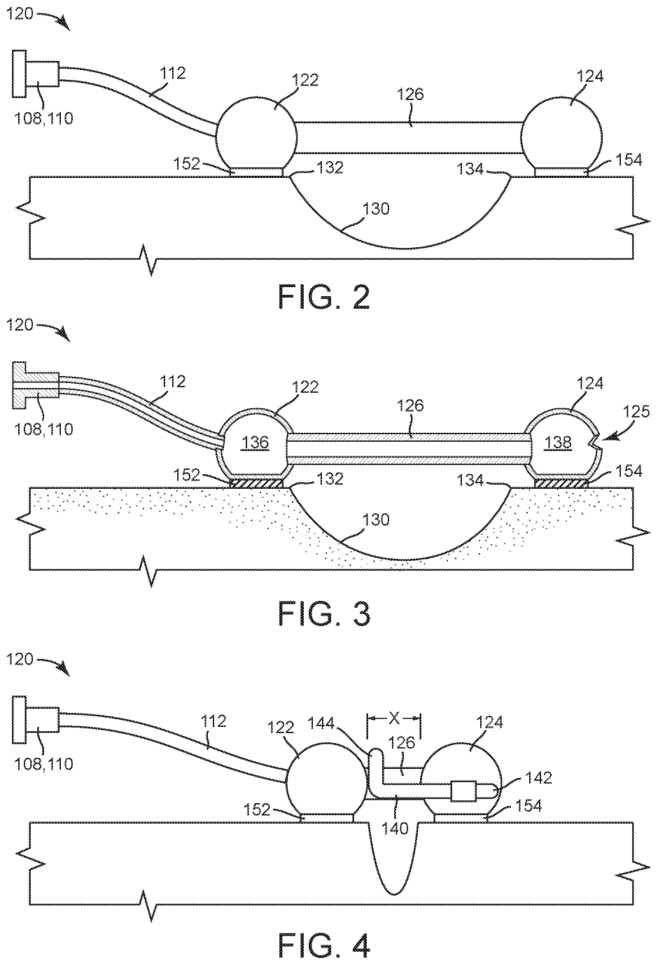

Referring now to FIGS. 2-4, a pneumatic module 120 is shown in greater detail, according to an exemplary embodiment. Pneumatic module 120 is shown to include a first node 122, a second node 124, and a pneumatic connector 126. Node 122 can be configured to attach to a patient's skin along a first edge 132 of wound 130. In some embodiments, first node 122 is attached to edge 132 via an adhesive pad 152. In other embodiments, first node 122 is attached to edge 132 via deep tissue hooks, using negative pressure, or any other means for securing node 122 to the patient's skin. Pneumatic tube 112 may attach to both first node 122 and one of ports 108-110 of pressure control device 102 and may form a pneumatic connection between first node 122 to the attached port 108-110.

Second node 124 can be configured to attach to the patient's skin along a second edge 132 of wound 130. In some embodiments, second node 124 is attached to edge 134 via an adhesive pad 154. In other embodiments, second node 124 is attached to edge 134 via deep tissue hooks, using negative pressure, or any other means for securing node 124 to the patient's skin. Pneumatic tube 112 may attach to both first node 122 and one of ports 108-110 of pressure control device 102 and may form a pneumatic connection between first node 122 to the attached port 108-110. Pneumatic connector 126 can be coupled to first node 122 and second node 124 and may form a pneumatic connection between first node 122 and second node 124. Pneumatic connector 126 can be configured to expand and contract based on the pneumatic pressure within pneumatic connector 126.

As shown in FIG. 3, first node 122 may be substantially hollow such that an empty space 136 exists within first node 122. Similarly, second node 124 may be substantially hollow such that an empty space 138 exists within second node 124. Spaces 136-138 may be pneumatically linked to each other via pneumatic connector 126. Space 136 may be pneumatically linked to one of ports 108-110 via pneumatic tube 112 such that the pneumatic pressure at port 108 or 110 is equivalent to the pneumatic pressure within space 136, within pneumatic connector 126, and within space 138. In some embodiments, one or more of nodes 122-124 may include a safety valve 125 to relieve pressure (e.g., positive pressure or negative pressure) within the corresponding pneumatic module 120 in the event of mechanical failure.

In some embodiments, pneumatic module 120 is attached to the patient's skin using negative pressure. For example, adhesive pads 152-154 can be replaced with suction cups configured to contact the patient's skin. The suction cups can be pneumatically linked to spaces 136-138 within nodes 122-124. Accordingly, the negative pressure within nodes 122-124 can be used to maintain suction within the suction cups to ensure that nodes 122-124 remain attached to the patient's skin.

As shown in FIG. 4, pneumatic module 120 may include an interlocking feature 140. Interlocking feature 140 may be a physical bracket (e.g., an L-bracket), bar, or other mechanical feature configured to maintain a minimum separation distance X between nodes 122 and 124. For example, a first end 142 of interlocking feature 140 may be attached to node 122. A second end 144 of interlocking feature 140 may be configured to contact node 124 when nodes 122-124 reach the minimum separation distance X to prevent nodes 122-124 from moving closer together. Alternatively, first end 142 of interlocking feature 140 may be attached to node 124 and second end 144 may be configured to contact node 122 when nodes 122-124 reach the minimum separation distance X. In some embodiments, interlocking feature 140 is adjustable to allow the minimum separation distance X to be increased or decreased.

### Device Operation

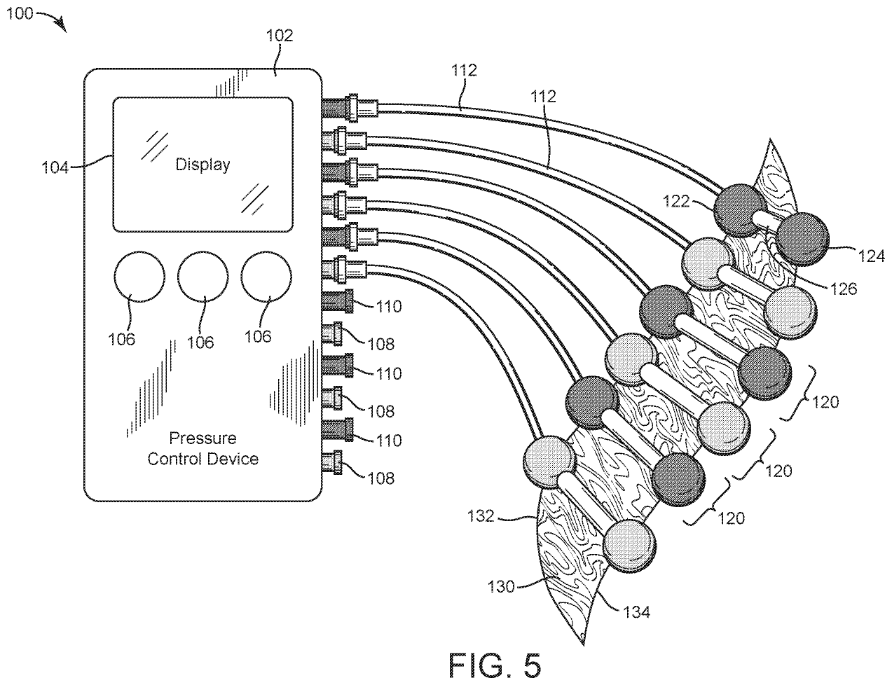

Referring now to FIGS. 5-7, several drawings illustrating the operation of pneumatic device 100 are shown, according to an exemplary embodiment. Pressure control device 102 is shown to include two sets of pneumatic ports 108 and 110. A first set of pneumatic modules 120 may be pneumatically coupled to ports 108, whereas a second set of pneumatic modules 120 may be pneumatically coupled to ports 110. In some embodiments, pressure control device 102 is configured to independently control the pneumatic pressure at each set of ports 108 and 110. For example, pressure control device 102 can simultaneously apply a first pneumatic pressure at ports 108 and a second pneumatic pressure, different from the first pneumatic pressure, at ports 110.

Advantageously, the ability to independently control the pneumatic pressure at each set of ports 108-110 allows pressure control device 102 to apply a different pneumatic pressure to each set of pneumatic modules 120. For example, pressure control device 102 can control the pneumatic pressure within the first set of pneumatic modules 120 by adjusting the pneumatic pressure at ports 108. Concurrently, pressure control device 102 can control the pneumatic pressure within the second set of pneumatic modules 120 by adjusting the pneumatic pressure at ports 110. This configuration allows pressure control device 102 to cause the pneumatic pressure within the first set of pneumatic modules 120 to be different from the pneumatic pressure within the second set of pneumatic modules 120 at any given moment in time.

A plurality of pneumatic modules 120 can be attached to the patient's skin along edges 132-134 of wound 130. For example, multiple nodes 122 can be attached to the patient's skin along edge 132, whereas multiple nodes 124 can be attached to the patient's skin along edge 134. Each of nodes 122 can be pneumatically coupled to one of ports 108-110 via a pneumatic tube 112 and to one of nodes 124 via a pneumatic connector 126. In some embodiments, pneumatic modules 120 in the first set and pneumatic modules 120 in the second set are arranged in alternating sequence along edges 132-134 of wound 130 (as shown in FIG. 5). For example, FIG. 5 shows six pneumatic modules 120 arranged in a linear sequence along edges 132-134. The first, third, and fifth pneumatic modules 120 in the linear sequence may be pneumatically coupled to ports 108, whereas the second, fourth, and sixth pneumatic modules 120 in the linear sequence may be pneumatically coupled to ports 110. In some embodiments, pneumatic modules 120 are arranged such that each pneumatic module 120 in the first set is separated from each other pneumatic module 120 in the first set by at least one pneumatic module 120 in the second set.

Pressure control device 102 can be configured to alternate pneumatic modules 120 between a first state and a second state. In the first state, the pneumatic pressure within the first set of pneumatic modules 120 may be less than the pneumatic pressure within the second set of pneumatic modules 120. In the second state, the pneumatic pressure within the first set of pneumatic modules 120 may be greater than the pneumatic pressure within the second set of pneumatic modules 120. The different pneumatic pressures within each set of pneumatic modules 120 may cause some pneumatic modules 120 to expand or relax while other pneumatic modules 120 contract.

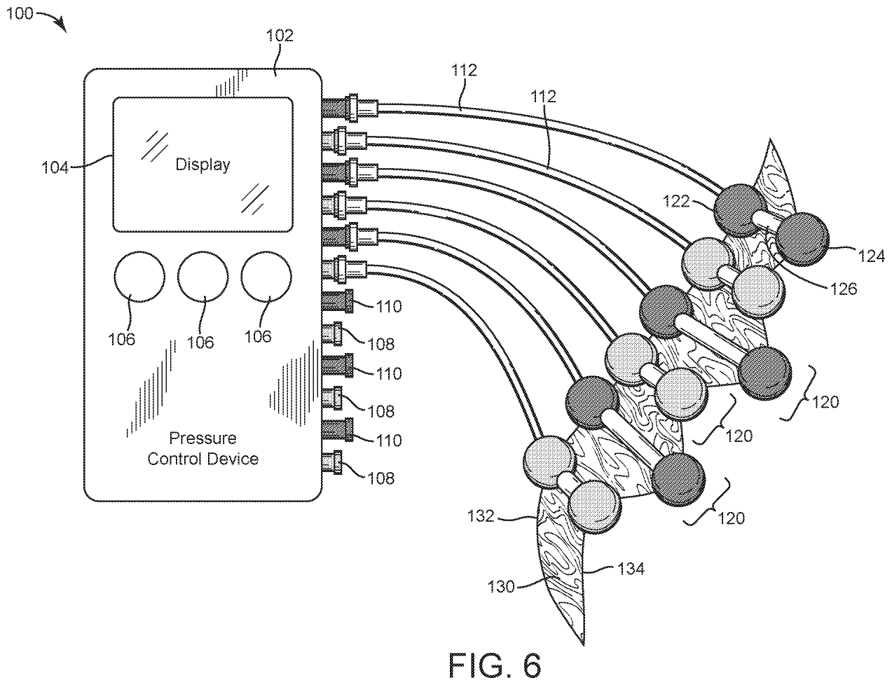

Advantageously, alternating pneumatic modules 120 between the first state and the second state may prevent skin breakdown by intermittently relaxing the stress applied to the patient's skin at the location of each pneumatic module 120. For example, in the first state, the first set of pneumatic modules 120 may held in a contracted position by maintaining the pneumatic pressure within the first set of pneumatic modules 120 at a negative pressure relative to ambient pressure. While the first set of pneumatic modules 120 are held in the contracted position, the second set of pneumatic modules 120 may be relaxed by adjusting the pneumatic pressure within the second set of pneumatic modules 120 to be close to ambient pressure. The position of pneumatic modules 120 in the first state is shown in FIG. 6. This allows pressure control device 102 to relax the stress applied to the patient's skin at the location of each pneumatic module 120 in the second set while ensuring that wound 130 does not reopen by maintaining each pneumatic module 120 in the first set in the contracted position.

Similarly, in the second state, the first set of pneumatic modules 120 be relaxed by adjusting the pneumatic pressure within the first set of pneumatic modules 120 to be close to ambient pressure. While the first set of pneumatic modules 120 relax, the second set of pneumatic modules 120 may held in a contracted position by maintaining the pneumatic pressure within the second set of pneumatic modules 120 at a negative pressure relative to ambient pressure. This allows pressure control device 102 to relax the stress applied to the patient's skin at the location of each pneumatic module 120 in the first set while ensuring that wound 130 does not reopen by maintaining each pneumatic module 120 in the second set in the contracted position. Alternating between the first state and the second state allows each of pneumatic modules 120 to intermittently relax to reduce the stress applied to the patient's skin to prevent skin breakdown at the location of each pneumatic module 120.

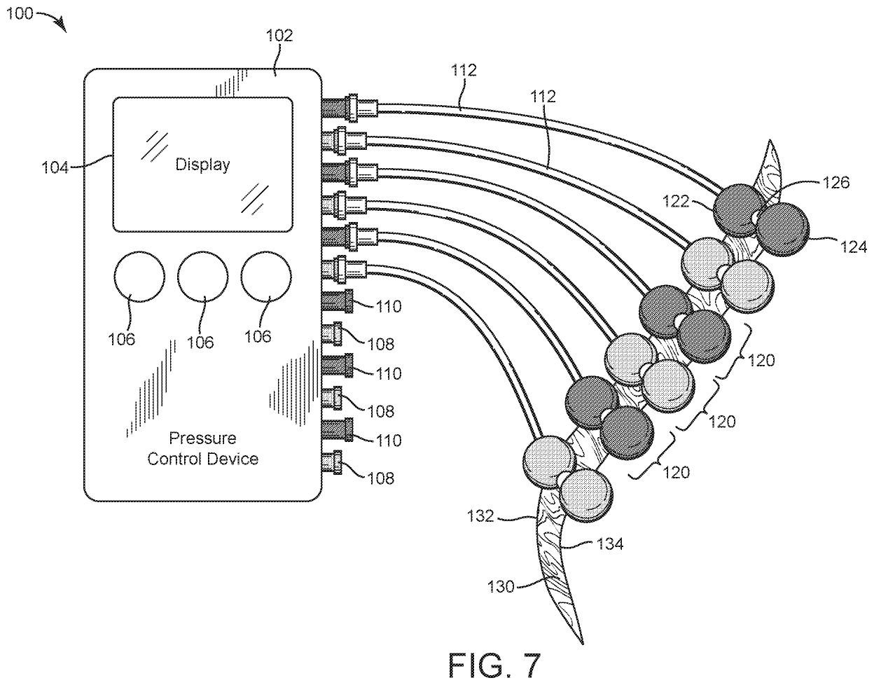

Pressure control device 102 can be configured to gradually reduce the pressure within each pneumatic module 120 to draw nodes 122-124 closer together and facilitate wound closure. For example, pressure control device 102 can reduce the pressure within each pneumatic module 120 until all pneumatic modules 120 reach the contracted positions shown in FIG. 7. Once pneumatic modules 120 are fully contracted, pneumatic modules 120 can be removed from the patients skin. If wound 130 needs to be reopened, pressure control device 102 can increase the pressure within each pneumatic module 120 to push nodes 122-124 apart and reopen wound 130. Pressure control device 102 can be used to both open and close wound 130 (e.g., an abdominal wound) in a controlled manner during ongoing care without the need for sutures.

In some embodiments, pressure control device 102 is configured to apply negative pressure wound therapy (NPWT). For example, pneumatic modules 120 may be attached to the patient's skin under a wound dressing or the wound dressing may be located between pneumatic modules 120 and the patient's skin. If pneumatic modules 120 are located under the wound dressing, a bespoke outlet can be used to seal the pneumatic connection to each pneumatic module 120. In some embodiments, the negative pressure applied to pneumatic modules 120 can also be applied to wound 130. For example, pneumatic modules 120 can be pneumatically coupled to a space between the wound dressing and the patient's skin and configured to provide negative pressure within the space to promote NPWT.

### Example Pneumatic Module and Manifold

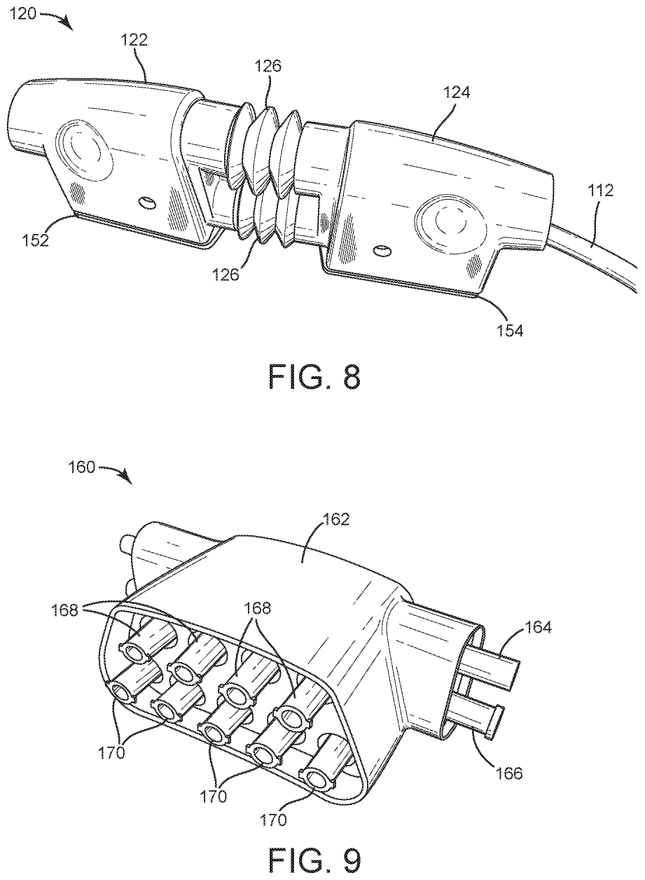

Referring now to FIG. 8, another example of a pneumatic module 120 is shown, according to an exemplary embodiment. In some embodiments, pneumatic module 120 includes multiple pneumatic connectors 126 that form a pneumatic connection between nodes 122 and 124. Each pneumatic connector 126 can be configured to expand when the pneumatic pressure within pneumatic module 120 is increased and contract when the pneumatic pressure within pneumatic module 120 is decreased. For example, pneumatic connectors 126 may be structural bellows configured to expand and contract linearly.

Referring now to FIG. 9, a manifold 160 is shown, according to an exemplary embodiment. Manifold 160 is shown to include two input ports 164 and 166 and a plurality of output ports 168 and 170. Input port 164 may be pneumatically connected to each of output ports 168, whereas input port 166 may be pneumatically connected to each of output ports 170.

Manifold 160 allows a single pneumatic tube 112 to connect to multiple pneumatic modules 120. For example, a single pneumatic tube 112 can be connected to port 108 of pressure control device and to input port 164 of manifold 160. Multiple pneumatic modules 120 (e.g., the first set of pneumatic modules 120) can be connected to output ports 168. Pressure control device 102 can control the pneumatic pressure within each of the first set of pneumatic modules 120 by adjusting the pressure at a single port 108 of pressure control device. Similarly, a single pneumatic tube 112 can be connected to port 110 of pressure control device and to input port 166 of manifold 160. Multiple pneumatic modules 120 (e.g., the second set of pneumatic modules 120) can be connected to output ports 170. Pressure control device 102 can control the pneumatic pressure within each of the second set of pneumatic modules 120 by adjusting the pressure at a single port 110 of pressure control device 102.

### Collapsible Foam Pneumatic Module

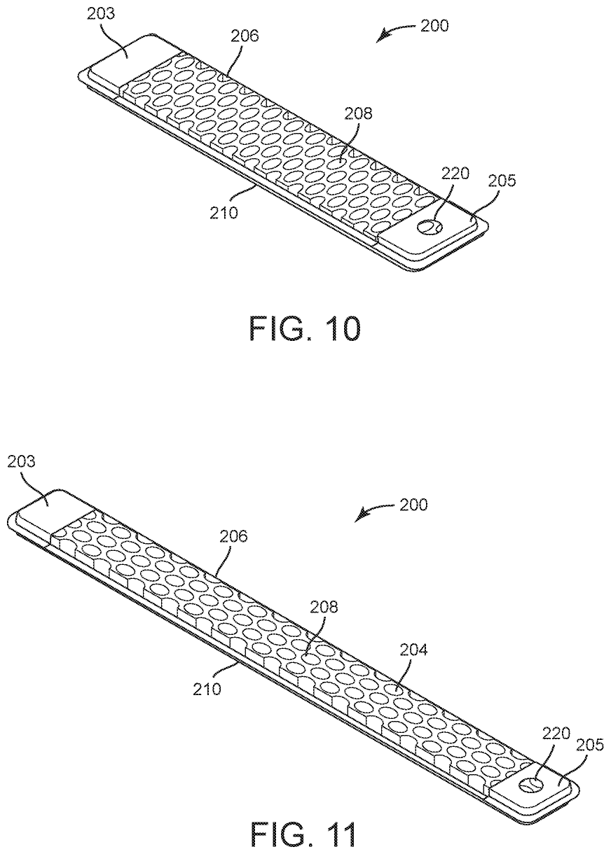

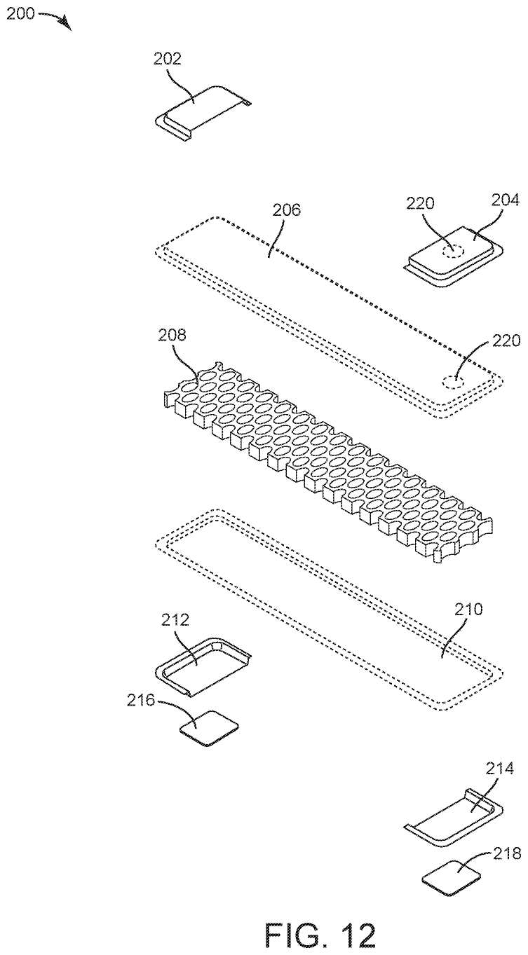

Referring now to FIGS. 10-13, another pneumatic module 200 is shown, according to an exemplary embodiment. Pneumatic module 200 can be used in combination with pressure control device 102 in place of pneumatic module 120. Pneumatic module 200 is shown to include a collapsible foam material 208 sealed between a top sealing film 206 and a bottom sealing film 210. Sealing films 206 and 210 may be substantially impermeable to air and may provide a sealed chamber within which collapsible foam material 208 is mounted. In some embodiments, sealing films 206 and 210 are polyurethane films. Collapsible foam material 208 may be configured to flex longitudinally. In some embodiments, collapsible foam material 208 is configured to expand and collapse longitudinally based on the pressure between sealing films 206 and 210.

In some embodiments, collapsible foam material 208 is applied to a patient's skin in a stretched state, shown in FIG. 11. Collapsible foam material 208 may be configured to collapse longitudinally upon application of a negative pressure between sealing films 206 and 210. The negative pressure may cause collapsible foam material 208 to return to a relaxed state, shown in FIG. 10. Similarly, collapsible foam material 208 may be configured to expand longitudinally upon application of a positive pressure between sealing films 206 and 210. The positive pressure may cause collapsible foam material 208 to expand to the stretched or expanded state. Pressure control device 102 may be pneumatically coupled to the space between sealing films 206 and 210 to control the expansion and collapse of foam material 208.

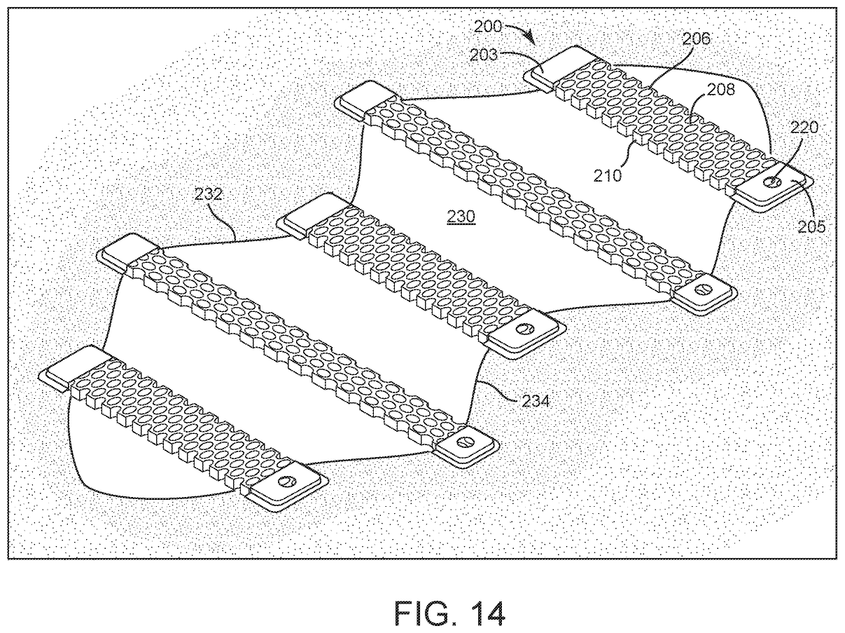

Pneumatic module 200 is shown to include a first foam clamp 203 having a top cap 202 and a bottom cap 212. In some embodiments, top cap 202 and bottom cap 212 are bonded or welded together to form first foam clamp 203. First foam clamp 203 may securely clamp a first end of collapsible foam material 208 between top cap 202 and bottom cap 212. An adhesive pad 216 can be attached to the bottom surface of bottom cap 212 and can be used to attach first foam clamp 203 to a patient's skin along a first edge 232 of a wound 230 (shown in FIG. 14).

Similarly, pneumatic module 200 is shown to include a second foam clamp 205 having a top cap 204 and a bottom cap 214. In some embodiments, top cap 204 and bottom cap 214 are bonded or welded together to form second foam clamp 205. Second foam clamp 205 may securely clamp a second end of collapsible foam material 208 between top cap 204 and bottom cap 214. An adhesive pad 218 can be attached to the bottom surface of bottom cap 214 and can be used to attach the second foam clamp to a patient's skin along a second edge 234 of a wound 230 (shown in FIG. 14).

Pneumatic module 220 can be configured to translate a pneumatic pressure into a linear force. For example, collapsible foam material 208 can be configured to extend and contract based on the pneumatic pressure between sealing films 206 and 210. When the pressure within pneumatic module 200 increases, collapsible foam material 208 may expand, thereby creating a linear force between foam clamps 203 and 205 which pushes foam clamps 203 and 205 apart from each other. Conversely, when the pressure within pneumatic module 200, collapsible foam material 208 may contract, thereby creating a linear force between foam clamps 203 and 205 which pulls foam clamps 203 and 205 toward each other. The linear forces created by collapsible foam material 208 may cause the edges of the wound to be pulled toward each other to close the wound or pushed apart from each other to open the wound.

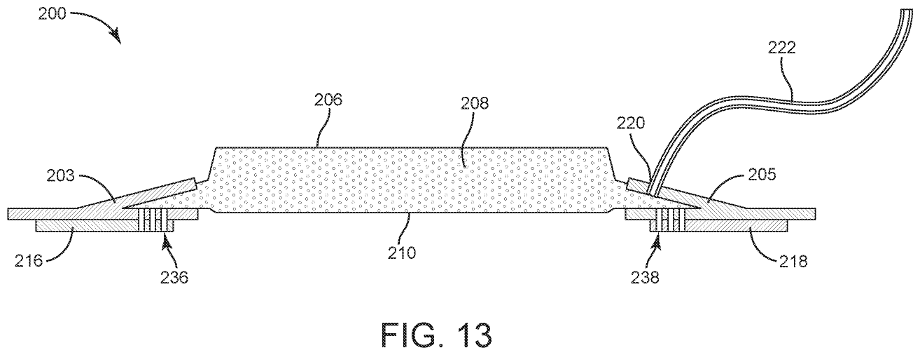

As shown in FIG. 13, pneumatic module 200 may include a port 220 in foam clamp 205. Port 220 may be configured to receive a pneumatic tube 222. Pneumatic tube 222 may be similar to pneumatic tube 112 or the same as pneumatic tube 112, as described with reference to FIGS. 1-10. For example, pneumatic tube 222 may pneumatically couple pneumatic module 200 to a port 108 or 110 of pressure control device 102. A first set of pneumatic modules 200 may be pneumatically coupled to ports 108, whereas a second set of pneumatic modules 200 may be pneumatically coupled to ports 110.

In some embodiments, foam clamp 203 includes a conduit 236 pneumatically coupled to both the patient's skin and to collapsible foam material 208. Similarly, foam clamp 205 may include a conduit 238 pneumatically coupled to both the patient's skin and to collapsible foam material 208. Conduits 236 and 238 may be maintained at an internal pressure less than an ambient pressure. For example, upon application of a negative pressure within collapsible foam material 208, the internal pressure within conduits 236 and 238 may decrease below ambient pressure. In some embodiments, foam clamps 203 and 205 are held to the patient's skin by the reduced pressure (i.e., the suction) within conduits 236 and 238.

### Device Operation

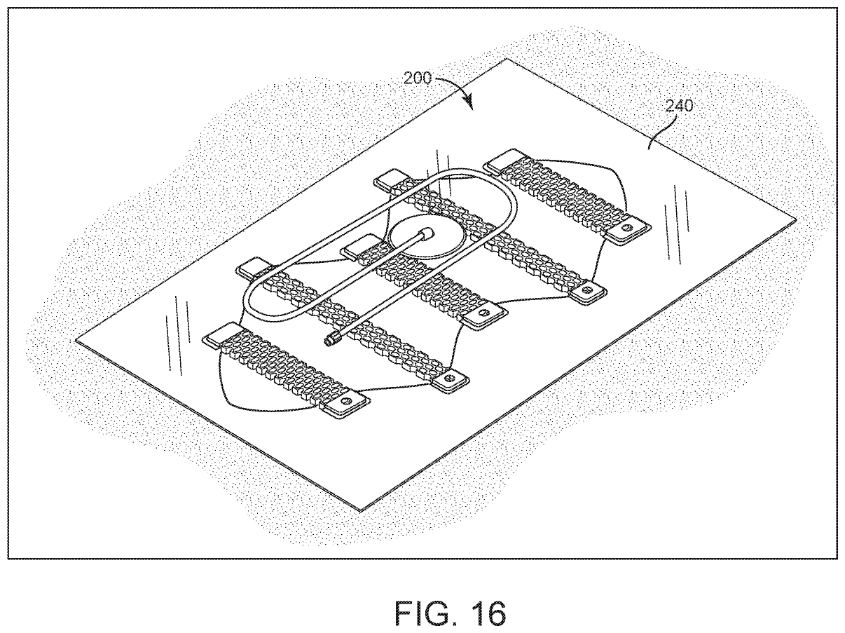

Referring now to FIGS. 14-16, a plurality of pneumatic modules 200 attached to a patient's skin are shown, according to an exemplary embodiment. Pneumatic modules 200 may include a first set of pneumatic modules 200 coupled to ports 108 of pressure control device 102 and a second set of pneumatic modules 200 coupled to ports 110 of pressure control device 102. Pressure control device 102 can adjust the pneumatic pressure within each pneumatic module 200 by increasing the pressure at the port 108 or 110 to which the pneumatic module 200 is coupled.

In some embodiments, pneumatic modules 200 in the first set and pneumatic modules 200 in the second set are arranged in alternating sequence along edges 232-234 of wound 230 (as shown in FIG. 13). For example, FIG. 13 shows five pneumatic modules 200 arranged in a linear sequence along edges 232-234. The first, third, and fifth pneumatic modules 200 in the linear sequence may be pneumatically coupled to ports 108, whereas the second and fourth pneumatic modules 200 in the linear sequence may be pneumatically coupled to ports 110. In some embodiments, pneumatic modules 200 are arranged such that each pneumatic module 200 in the first set is separated from each other pneumatic module 200 in the first set by at least one pneumatic module 200 in the second set.

Pressure control device 102 can be configured to alternate pneumatic modules 200 between a first state and a second state. In the first state, the pneumatic pressure within the first set of pneumatic modules 200 may be less than the pneumatic pressure within the second set of pneumatic modules 200. In the second state, the pneumatic pressure within the first set of pneumatic modules 200 may be greater than the pneumatic pressure within the second set of pneumatic modules 200. The different pneumatic pressures within each set of pneumatic modules 200 may cause some pneumatic modules 200 to expand or relax while other pneumatic modules 200 contract.

Advantageously, alternating pneumatic modules 200 between the first state and the second state may prevent skin breakdown by intermittently relaxing the stress applied to the patient's skin at the location of each pneumatic module 200. For example, in the first state, the first set of pneumatic modules 200 may held in a contracted position by maintaining the pneumatic pressure within the first set of pneumatic modules 200 at a negative pressure relative to ambient pressure. While the first set of pneumatic modules 200 are held in the contracted position, the second set of pneumatic modules 200 may be relaxed by adjusting the pneumatic pressure within the second set of pneumatic modules 200 to be close to ambient pressure. The position of pneumatic modules 200 in the first state is shown in FIG. 13. This allows pressure control device 102 to relax the stress applied to the patient's skin at the location of each pneumatic module 200 in the second set while ensuring that wound 230 does not reopen by maintaining each pneumatic module 200 in the first set in the contracted position.

Similarly, in the second state, the first set of pneumatic modules 200 be relaxed by adjusting the pneumatic pressure within the first set of pneumatic modules 200 to be close to ambient pressure. While the first set of pneumatic modules 200 relax, the second set of pneumatic modules 200 may held in a contracted position by maintaining the pneumatic pressure within the second set of pneumatic modules 200 at a negative pressure relative to ambient pressure. This allows pressure control device 102 to relax the stress applied to the patient's skin at the location of each pneumatic module 200 in the first set while ensuring that wound 230 does not reopen by maintaining each pneumatic module 200 in the second set in the contracted position. Alternating between the first state and the second state allows each of pneumatic modules 200 to intermittently relax to reduce the stress applied to the patient's skin to prevent skin breakdown at the location of each pneumatic module 200.

Pressure control device 102 can be configured to gradually reduce the pressure within each pneumatic module 200 to draw the foam clamps closer together and facilitate wound closure. For example, pressure control device 102 can reduce the pressure within each pneumatic module 200 until all pneumatic modules 200 reach the collapsed position. Once pneumatic modules 200 are fully collapsed, pneumatic modules 200 can be removed from the patients skin. If wound 230 needs to be reopened, pressure control device 102 can increase the pressure within each pneumatic module 200 to push the foam clamps apart and reopen wound 230. Pressure control device 102 can be used to both open and close wound 230 (e.g., an abdominal wound) in a controlled manner during ongoing care without the need for sutures.

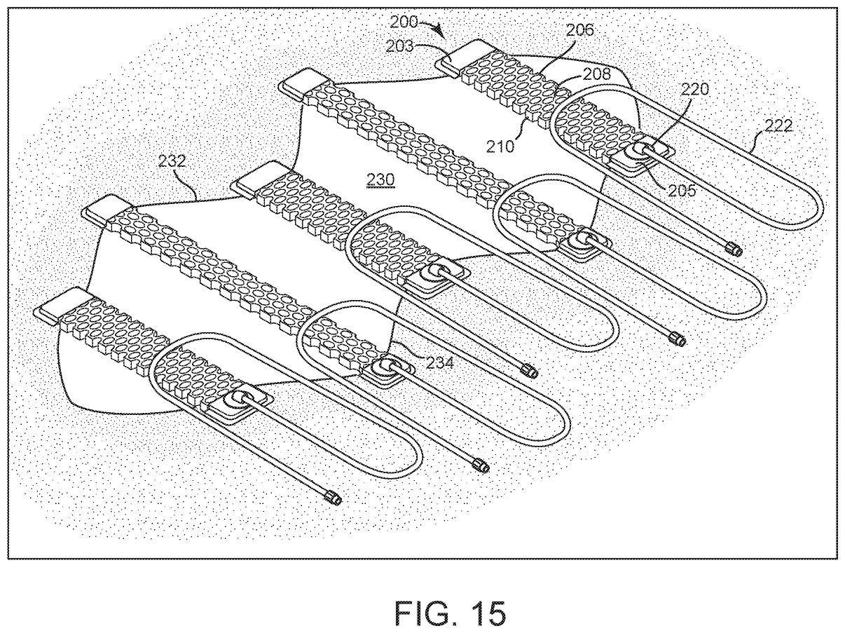

In some embodiments, pressure control device 102 is configured to apply negative pressure wound therapy (NPWT). For example, FIG. 15 shows a plurality of pneumatic modules 200 attached to the patient's skin under a wound dressing 240. Wound dressing 240 may be substantially impermeable to air such that a sealed space exists between wound 230 and the ambient environment. In some embodiments, the negative pressure applied to pneumatic modules 200 can also be applied to wound 230. For example, pneumatic modules 200 can be pneumatically coupled to the space between wound dressing 240 and wound 230 and configured to provide negative pressure within the space to promote NPWT.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A device comprising:
a plurality of pneumatic modules configured to attach to a patient's skin along edges of a wound and to translate a pneumatic pressure into a linear force, each pneumatic module configured to move between an extended position and a contracted position based on the pneumatic pressure within the pneumatic module; and
a pressure control device configured to be pneumatically coupled to each of the pneumatic modules and configured to alternate the plurality of pneumatic modules between:
a first state in which the pneumatic pressure within a first set of the pneumatic modules is less than the pneumatic pressure within a second set of the pneumatic modules; and
a second state in which the pneumatic pressure within the first set of the pneumatic modules is greater than the pneumatic pressure within the second set of the pneumatic modules.

2. The device of claim 1, wherein the pressure control device is configured to apply a different pneumatic pressure to each of the plurality of pneumatic modules.

3. The device of claim 1, wherein the pressure control device is configured to be pneumatically coupled to a space between the wound and a wound dressing and configured to apply a reduced pressure within the space between the wound and the wound dressing.

4. The device of claim 1, wherein the first set of pneumatic modules and the second set of pneumatic modules are arranged in an alternating sequence along the edges of the wound.

5. The device of claim 1, wherein the first set of pneumatic modules and the second set of pneumatic modules are arranged along the edges of the wound such that each pneumatic module in the first set is separated from each other pneumatic module in the first set by at least one pneumatic module in the second set.

6. The device of claim 1, wherein:
reducing the pneumatic pressure within a pneumatic module causes the pneumatic module to move toward the contracted position; and
increasing the pneumatic pressure within a pneumatic module causes the pneumatic module to move toward the extended position.

7. The device of claim 1, wherein reducing the pneumatic pressure within a pneumatic module increases the linear force exerted by the pneumatic module and is configured to increase a stress applied to the patient's skin by the pneumatic module.

8. The device of claim 1, wherein increasing the pneumatic pressure within a pneumatic module relaxes the linear force exerted by the pneumatic module and is configured to reduce a stress applied to the patient's skin by the pneumatic module.

9. The device of claim 1, wherein one or more of the pneumatic modules comprises a relief valve configured to vent the pneumatic module when a difference between ambient pressure and the pneumatic pressure within the pneumatic module exceeds a relief pressure threshold.

10. The device of claim 1, further comprising a plurality of pneumatic tubes connecting the pressure control device to the plurality of pneumatic modules.

11. The device of claim 1, wherein each pneumatic module comprises:
a first node configured to attach to the patient's skin along a first edge of the wound;
a second node configured to attach to the patient's skin along a second edge of the wound; and
a pneumatic connector coupled to the first node and the second node and forming a pneumatic connection between the first node and the second node, the pneumatic connector configured to extend and contract based on the pneumatic pressure within the pneumatic module.

12-16. (canceled)

17. A device comprising:
a plurality of pneumatic modules, each pneumatic module comprising:
a first node configured to attach to a patient's skin along a first edge of the wound;
a second node configured to attach to the patient's skin along a second edge of the wound; and
a pneumatic connector coupled to the first node and the second node and forming a pneumatic connection between the first node and the second node, the pneumatic connector configured to extend and contract based on a pneumatic pressure within the pneumatic module; and
a pressure control device configured to be pneumatically coupled to each of the pneumatic modules and configured to alternate the plurality of pneumatic modules between:
a first state in which the pneumatic pressure within a first set of the pneumatic modules is less than the pneumatic pressure within a second set of the pneumatic modules; and
a second state in which the pneumatic pressure within the first set of the pneumatic modules is greater than the pneumatic pressure within the second set of the pneumatic modules.

18. The device of claim 17, wherein each node comprises an adhesive pad configured to adhere the node to the patient's skin.

19. The device of claim 17, wherein each node comprises a deep tissue hook configured to attach the node to the patient's skin.

20. The device of claim 17, wherein:
each node comprises an internal volume pneumatically connected to the patient's skin and maintained at an internal pressure less than an external pressure outside the node; and
each node is held to the patient's skin by a pressure differential between the internal pressure and the external pressure.

21. The device of claim 17, wherein one or more of the pneumatic modules comprises an interlocking feature configured to engage when a separation distance between the first node and the second node reaches a minimum distance threshold and to prevent the separation distance from decreasing below the minimum distance threshold.

22. The device of claim 21, wherein the interlocking feature is adjustable to allow the minimum distance threshold to be increased or decreased.

23-31. (canceled)

32. A device comprising:
a first foam clamp configured to attach to a patient's skin along a first edge of the wound;
a second foam clamp configured to attach to the patient's skin along a second edge of the wound; and
a foam material disposed between and coupled to the first foam clamp and the second foam clamp, the foam material being longitudinally collapsible to draw the first and second foam clamps toward each other upon application of a negative pressure.

33. The device of claim 32, wherein the foam material is longitudinally expandable to push the first and second foam clamps away from each other upon application of a positive pressure.

34. The device of claim 32, wherein the foam material is configured to return to a non-collapsed state and push the first and second foam clamps away from each other upon removal of the negative pressure.

35. The device of claim 32, further comprising a pressure control device pneumatically coupled to the foam material and configured to control a pressure within the foam material to cause the foam material to draw the first and second foam clamps toward each other.

36. The device of claim 32, further comprising an impermeable layer along an outer surface of the foam material and configured to prevent airflow between the foam material and an ambient environment.

37. (canceled)

38. The device of claim 32, further comprising an impermeable layer along an wound-facing surface of the foam material and configured to prevent airflow between the foam material and the wound.

39. The device of claim 32, wherein the foam material is pneumatically coupled to a space between the wound and a wound dressing such that the pneumatic pressure within the foam material is applied to the space between the wound and the wound dressing.

40. The device of claim 32, wherein the foam clamps comprise conduits pneumatically connected to the patient's skin and maintained at an internal pressure less than an ambient pressure; and
the foam clamps are held to the patient's skin by a pressure differential between the internal pressure and the ambient pressure.

41-42. (canceled)

43. The device of claim 32, wherein one or more of the foam clamps comprises an interlocking feature configured to engage when a separation distance between the first foam clamp and the second foam clamp reaches a minimum distance threshold and to prevent the separation distance from decreasing below the minimum distance threshold.

44. The device of claim 43, wherein the interlocking feature is adjustable to allow the minimum distance threshold to be increased or decreased.

45. The device of claim 32, wherein:
reducing the pressure within the foam material causes the foam material to draw the first and second foam clamps toward each other; and
increasing the pressure within the foam material causes the foam material to push the first and second foam clamps away from each other.

46-48. (canceled)
